(19) United States
(12) Patent Application Publication
(10) Pub. No.: US 2010/0136675 A1
(43) Pub. Date: Jun. 3, 2010

(54) Incubation and Dry Storage Apparatus

(75) Inventors: Hubert Heeg, Aschaffenburg (DE); Wolfgang Rether, Hasselroth (DE)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: THERMO ELECTRON LED GMBH, Langenselbold (DE)

(21) Appl. No.: 12/628,891

(22) Filed: Dec. 1, 2009

(30) Foreign Application Priority Data
Dec. 1, 2008 (DE) DE 102008059854.2

(51) Int. Cl. C12M 1/00

(52) U.S. Cl. 435/303.1

(57) ABSTRACT

An incubation and dry storage apparatus, especially for samples made of organic material, is provided.

### CROSS-REFERENCE TO RELATED APPLICATIONS

This application claims priority to foreign Patent Application DE 10 2008 059854.2, filed on Dec. 1, 2008, the disclosure of which is incorporated herein by reference in its entirety.

### FIELD OF THE INVENTION

The invention relates to an incubation and dry storage apparatus, especially for samples made of organic material.

### BACKGROUND OF THE INVENTION

Incubation apparatuses on the one hand and dry storage apparatuses on the other hand are used in biology and medicine for example to store organic materials under defined exterior conditions.

Incubation apparatuses are widely used for breeding and storing microorganisms which can strongly accelerate their growth under special living conditions. Generally, the temperature and relative air humidity as important growth parameters are held constant at a specific level over a prolonged period of time in such apparatuses. These mostly concern temperatures which correspond roughly to the body temperature of humans, i.e. preferably approx. 37° C. The relative humidity of air is preferably set and held above 90% RH in a conventional incubation apparatus, since this represents optimal conditions for growth for most microorganisms. The incubation apparatuses are therefore equipped with a water reservoir and a respective humidifying device. Even when removing the water from the reservoir and cutting off the humidifying, no relative humidity values beneath approx. 30% to 40% RH can be achieved in such incubation apparatuses.

In contrast to the incubation apparatuses, dry storage and a respective apparatus for dry storage are used for the purpose of storing substrates of various kinds and shall offer optimal protection for storing high-quality components and various substrates in a particle-free, germ-free and corrosion-free manner. For this purpose, ambient parameters are preferably used which lie at relatively low temperatures of preferably approx. 4 to 10° C. and at a low relative humidity of air in the range of under 10% RH. In contrast to the incubation apparatuses, the absorption of humidity by the substrates should be minimized.

Temperable incubation apparatuses or dry storage devices for storing tissue samples, cell cultures, substrates, etc. are used in the pharmaceutical industry for example.

In this respect it is very important that these apparatuses are very easy to clean for hygienic reasons. This is especially necessary in order to prevent transmission of germs and undesirable infections. Conventional dry storage apparatuses are not sufficiently equipped in order to prevent the likelihood of contamination and to offer favorable cleaning capability according to the medical requirements which also apply to incubation operation.

Various basic forms of incubation apparatuses on the one hand and dry storage apparatuses on the other hand are known in the state of the art. One can distinguish between the following systems in dry storage apparatuses.

### Dehumidification by Circulating Air

A portion of the indoor air is guided via a bypass to the dehumidifier arranged internally in the appliance and is optionally cooled before the dehumidified and optionally cooled air is supplied to the interior space of the dry storage apparatus. This variant of air dehumidifying is recommended especially for larger storage chambers, cabinets or shelf systems, where the ambient air is dried according to the adsorption principle.

Purging with Nitrogen or Dried Air

Such a system is especially used in the electronics industry for dry storage of sensitive SMD components and provides a controlled supply especially of nitrogen into the interior space of the storage apparatus in order to produce a non-corrosive storage atmosphere. Two types of air dryers are used. Compressed-air dryers which work according to the adsorption principle are used for smaller cabinets or acrylic boxes for example. In this case, compressed air from a provided compressor or from an in-house network is filtered, dried and then introduced into the storage chambers. A circulating-air system is preferable for larger systems, as described above, in which an adsorption dryer can also be used.

Different variants of purging can be performed depending on the size of the system and the requirements. In addition to permanent purging, the systems are also occasionally operated with automatic quick or permanent purging or with demand-controlled purging.

A relative air humidity of <5% is achieved in both variants for example. In order to prevent the penetration of air humidity into the chamber system, there is always a slight overpressure in the chambers.

The disadvantage of these known solutions is that they generally have no devices which also set and hold the air humidity constantly over a wide range. Therefore, no increased relative humidity values of larger than 90% RH can be achieved for example with the devices. Moreover, the temperature ranges of the dry storage apparatuses are configured to the intended low temperatures of about 5° C. and tempering to values larger than 35° C. is often not provided. Moreover, the known dry storage apparatuses are not configured especially with respect to the high requirements of hygiene and for averting the likelihood of contamination in such a way that they would be adequate in an operation for the incubation of medical samples for example.

### SUMMARY OF THE INVENTION

Embodiments of the present invention advantageously arrange an incubation and dry storage apparatus in such a way that it is suitable for tempering samples of organic material and is further easy to clean in the interior space. At the same time, constant adherence of the temperature and air humidity over a wide range in the interior space is to be ensured and a possibility is to be offered to provide both functions of dry storage on the one hand and incubation on the other hand in one single apparatus.

Embodiments of the present invention provide an incubation and dry storage apparatus that comprises an incubation unit with a heatable and coolable interior space and a dehumidifying unit, with the dehumidifying unit being connected with the interior space of the incubation unit via at least one connection means in such a way that the dehumidifying unit can be connectible to and disconnectible from the interior space.

Furthermore, means are provided, in accordance with embodiments of the present invention, for refrigerating and heating the interior space of the apparatus over a wide range of less than 4° C. and higher than 60° C.

By using a dehumidifying unit which can be connected to an incubation unit, the required setting and keeping constant of the relative air humidity can be favorably achieved over a range of less than 5% RH and more than 90% RH in the interior of the apparatus.

The combined dry storage and incubation apparatus can be used advantageously, especially in the storing and subsequent incubation of medical samples, because the apparatus can combine both functions as a result of the connectible dehumidifying unit. The utilization of two separate devices as previously required in the state of the art can therefore be avoided. Embodiments of the present invention use one device for the two operating states: (a) warm and humid, and (b) cold and dry. The transport of samples from a dry storage apparatus to an incubation apparatus, and the resulting likelihood of contamination, can advantageously be avoided. Moreover, embodiments of the present invention offer favorable cleaning capabilities and a configuration that corresponds to medical requirements for preventing contaminations.

### BRIEF DESCRIPTION OF THE DRAWINGS

Further details, features and advantages of the invention are obtained from the following description of embodiments by reference to the relevant schematic drawing, wherein:

FIG. 1 shows a side view of an incubation and dry storage apparatus, according to an embodiment of the present invention.

### DETAILED DESCRIPTION

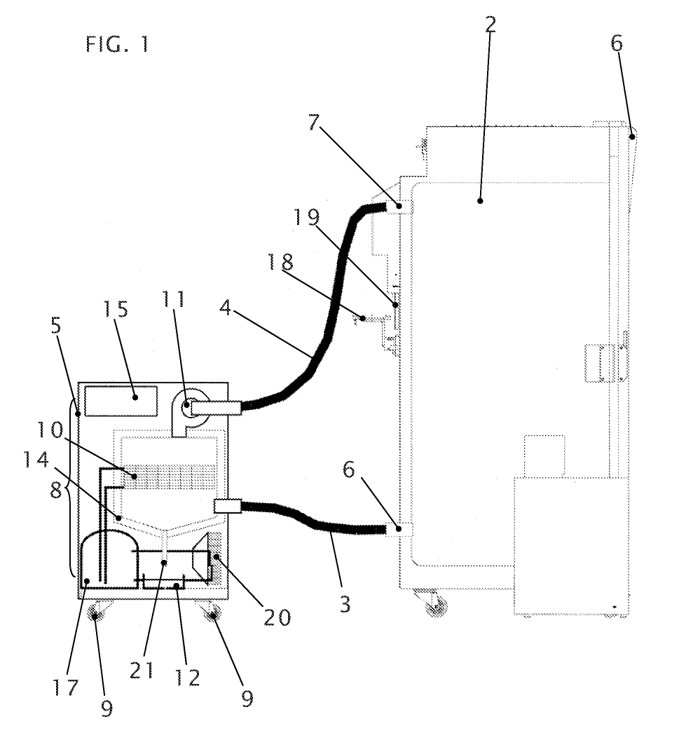

FIG. 1 shows a conceptual illustration of an embodiment in accordance with the invention of an incubation and dry storage apparatus 1. Samples can be introduced into the interior space 2 of the incubation unit 6 via a sample receiving system 18 which conveys the samples such as microtiter plates from the outside into the interior space via the access flap 19. The interior space 2 of the incubation unit 6 is formed by inner boundary areas, with feeding and discharging elements 3 and 4 being arranged on the rear side of the incubation unit 6 which are connected with the dehumidifying unit 5. The connection can preferably be provided via a flange system 7. The flange system 7 can optionally be arranged in such a way that it comprises the activation and deactivation of dehumidifying device 5 which is external in the illustrated embodiment of the invention towards the interior space 2 of the incubation unit 6. The feeding and discharging elements 3 and 4 can advantageously be arranged as a tube and/or hose connection, with the feeding element 3 being arranged to be heatable. Any undesirable increased temperature gradient in the interior space 2 can thus be avoided.

In incubation operation, water is filled into the incubation unit 6 for humidifying and the desired incubation temperature of 37° C. for example is set. The dehumidifying unit 5 is switched off in this operating mode. A relative humidity of more than 90% RH can be obtained in the interior space 2.

It is advantageous to use an external thermostat with refrigerating and heating function or an internal heating device and optionally an external refrigerating device for tempering the incubation and dry storage apparatus. The external components must be provided for this purpose with the required connections and a container for receiving the heat carrier and a pump. Furthermore, the external components are provided with a separate temperature control.

The heat carrier system is advantageously arranged as an open or expandable system in an incubation and dry storage apparatus with an external refrigerating system. This allows connecting further devices, etc. to the external refrigerating system via respectively suitable connections, so that refrigerating capacity reserves of the refrigerating system can especially be used for other applications.

The transparent areas such as glass or acrylic glass panes can be provided with a heatable configuration in order to prevent the formation of condensate by air humidity on inspection windows of the incubation and dry storage apparatus.

A mixture of water and glycol can preferably be used as a heat carrier for the incubation and dry storage apparatus in accordance with the invention.

When the device is used as a dry storage, the humidifying water is removed from the incubation unit 6. The dehumidifying unit 5 is activated and the desired storage temperature of 4° C. for example is set in the incubation unit. The excessive humidity is frozen out of the interior space of the incubation unit in the dehumidifying unit 5 via a cold trap 8 with dew point control. A specific relative residual humidity value can be set precisely by the dew point control of the cold trap 8. The dehumidifying unit advantageously comprises a respective controller device 15 for this purpose. The temperature of the cold trap 8 is less than −22.5° C. at temperature of 4° C. and a desired residual humidity of less than 10% RH. The cold trap 8 can be arranged for example as a compressor refrigerating system with a refrigerating compressor 17, a heat exchanger 20 and a Cu evaporator 10. A fan system 11 can remove the cooled air from the interior space 2 of the incubation unit 6 via a discharge tube 4 and can guide the same over the cold trap in the dehumidifying unit 5 where it is dehumidified. Preferably, the air drawn for dehumidifying can flow through the cold trap 8 from the top to bottom. The return of the cooled, dehumidified air of the interior space from the dehumidifying unit 6 can preferably occur via a feed tube 3 which is heatable. An undesirably strong temperature gradient in the interior space 2 can thus be avoided. The dehumidifying unit 5 can comprise a defrosting routine for defrosting the evaporator 10. The water collected in this process can be discharged advantageously downwardly out of the refrigerating chamber 14 and be collected in a canister 12 via a drain 21. The canister 12 can be removed easily by the user. In order to prevent the thawed water from freezing on the walls of the refrigerating chamber 14, the floor area can advantageously be arranged as a funnel and can especially be heated.

The dehumidifying unit 5 can be advantageously connected in a detachable way with the interior space of the incubation unit 5. The dehumidifying unit 5 which is thus arranged as an external unit can be used for several incubation units. As a result, a dehumidifying unit 5 can advantageously form an incubation and dry storage apparatus especially for samples of organic material with one or several incubation units 6.

The dehumidifying unit 5 can further be equipped with rollers 9 for flexible use in the laboratory.

The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention.

What is claimed is:

1. An incubation and dry storage apparatus for organic material samples, comprising:
an incubation unit with a heatable and coolable interior space; and
a dehumidifying unit connected to the interior space of the incubation unit via at least one connection means in such a way that the dehumidifying unit is connectible to, and disconnectible from, the interior space.

2. An incubation and dry storage apparatus according to claim 1, wherein devices for connecting and disconnecting the dehumidifying unit are arranged in the connection means.

3. An incubation and dry storage apparatus according to claim 1, wherein the connection means is arranged as a flange system.

4. An incubation and dry storage apparatus according to claim 1, wherein the connection means is arranged between the interior space of the incubation unit and the dehumidifying unit as a feed element and a discharge element that are each connectible via a flange system.

5. An incubation and dry storage apparatus according to claim 4, wherein the feed element and the discharge element are arranged as tube and/or hose connection, with the feed element being heatable.

6. An incubation and dry storage apparatus according to claim 1, wherein a cold trap with dew point control is provided in the dehumidifying unit.

7. An incubation and dry storage apparatus according to claim 1, wherein the dehumidifying unit is detachably connected to the incubation unit.

8. An incubation and dry storage apparatus according to claim 1, wherein rollers are arranged on the dehumidifying unit.
